(19) United States
(12) Patent Application Publication
(10) Pub. No.: US 2012/0289841 A1
(43) Pub. Date: Nov. 15, 2012

(54) APPARATUS AND CALIBRATION METHOD FOR BLOOD PRESSURE MEASUREMENT

(75) Inventors: Yunquan Chen, Delta (CA); Yun Xu, Beijing (CN)

(73) Assignee: QDevice Medical, Inc., Delta (CA)

(21) Appl. No.: 13/511,555

(22) PCT Filed: Dec. 1, 2009
(86) PCT No.: PCT/CN09/75244

§ 371 Date: May 23, 2012

(51) Int. Cl. A61B 5/022

(52) U.S. Cl. 600/490

(57) ABSTRACT

Apparatus and a calibration method for measurement of blood pressure are used to determine personal PIP's for a given subject and to store the personal PIP's for future personal use in blood pressure measurement in the subject. The apparatus comprises at least one button communicating with a MPU unit for entering information used to determine the personal PIP's of the subject. The method comprises the steps of obtaining information required to determining the PIP's of a subject, determining the personal PIP's of the subject, and storing the personal PIP's of the subject for future personal use for the subject.

### TECHNICAL FIELD

The present invention relates to non-invasive methods for determining the blood pressure of a subject. More particularly, the invention relates to an improved method and apparatus for making oscillometric measurements of blood pressure.

### BACKGROUND

There is a variety of techniques for measuring blood pressure of a subject. The auscultatory method is considered the “gold” standard of blood pressure measurement. It involves inflating a cuff placed around a limb of the subject. Following inflation of the cuff, the cuff is permitted to deflate. Systolic blood pressure (“SBP”) is taken to be the cuff pressure at which Korotkoff sounds begin to occur as the cuff is deflated. Diastolic blood pressure (“DBP”) is taken to be the cuff pressure at which the Korotkoff sounds become muffled or disappear. The auscultatory method requires an experienced operator to make a judgment as to when the Korotkoff sounds start and when they stop.

One widely used method in automated electronic blood pressure monitors is the oscillometric method. In this method, the cuff of the oscillometric blood pressure monitor is first inflated to above the systolic pressure of the subject, then, deflated slowly and the pressure within the cuff is continuously monitored. As the cuff is deflated slowly, the pressure within the cuff exhibits a certain pressure versus time waveform. The waveform can be separated into two components, a decaying component representing the cuff pressure without the effect of pulse pressure and an oscillating component caused by blood pressure pulses in underlying arteries. The oscillating component may be represented by a curve known by those in the art as the “oscillometric envelope”, which starts at a low value when the cuff is inflated to a level beyond the subject's systolic SBP and then increases to a peak value P2 as the cuff pressure is reduced. Once the envelope has reached P2, the envelope then decays as the cuff pressure continues to decrease. SBP may be determined as the cuff pressure corresponding to the amplitude on the systolic side (before peak amplitude P2) of the oscillometric envelope which is equal to a certain fixed percentage of the peak amplitude P2. This percentage is known by those skilled in the art as the systolic Parameter Identification Point (“PIP”), and is generally considered to be about 55%. Similarly, DBP may be determined as the cuff pressure corresponding to the amplitude on the diastolic side (after peak amplitude P2) of the oscillometric envelope which is equal to a certain fixed percentage of the peak amplitude P2. This percentage is known as the diastolic PIP, which is generally considered to be close to 72%.

It is also possible to apply the oscillometric method during an inflation phase instead of the deflation phase of the measurement cycle. In this case. the cuff is inflated slowly and pressure in the cuff is continuously monitored in the slow inflation phase to determine the SBP and DBP.

Chen et al. have discovered that the above fixed PIP method results in statistical bias in SBP readings when a subject's SBP is higher than 140 mmHg, and address the problem in U.S. Pat. No. 6,719,703 “Method and apparatus for measuring blood pressure by the oscillometric technique”.

They disclose a method for using a variable systolic PIP based on the blood pressure of the subject.

We have found in our research that the oscillometric method results in large BP measurement errors (>10 mmHg) for about ten percent of the population compared with the auscultatory method. Therefore, it is desirable to further improve the accuracy of the oscillometric method.

### SUMMARY OF INVENTION

The present invention provides an improved method and apparatus for measuring BP in a subject, and in particular, an electronic BP monitor and method for calibrating the BP monitor for personal use for a given subject.

A method according to a basic embodiment of the invention comprises the steps of: determining a personal. PIP for a given subject and storing said personal PIP in said BP monitor for use in future measurement of the BP in said subject. Said personal PIP may be a personal systolic PIP or a personal diastolic PIP.

In a more detailed embodiment of the method, a cuff of an electronic BP monitor is placed around the limb of a subject for whom the BP monitor is to be calibrated to obtain the BP of the subject using the oscillometric technique and standard PIP's that apply to every subject without individual calibration. The BP obtained this way is called the standard oscillometric BP. During the slow deflation phase of the measurement cycle, an operator uses a stethoscope to determine the BP of the subject using the auscultatory method. The BP obtained this way is called the auscultatory BR The auscultatory SBP and DBP are entered through two or three buttons into the microprocessor unit (MPU) of the BP monitor so that a program embedded in the MPU in the BP monitor first finds the systolic time t1 and diastolic t3 at which the cuff pressure equals to the auscultatory SBP and DBP, respectively, and then finds amplitude P1 and P3 on the oscillometric envelope at the time t1 and t3, respectively. The program in the MPU calculates the personal systolic PIP, which is P1/P2 where P2 is the peak amplitude of the oscillometric envelope, and the personal diastolic PIP, which is P3/P2, for the subject, and stores the personal systolic and diastolic PIP's for future measurement of BP in the subject.

In an alternative embodiment of the method, the systolic time t1 and diastolic time t3 are obtained from a systolic and a diastolic signal entered by the operator by pressing one button in sequence or a systolic button and a diastolic button in sequence when the Korotkoff sounds start and stop in sequence during the deflation phase of the measurement cycle.

In another embodiment of the method, a slow inflation phase is used for measurement of BP instead of the commonly use slow deflation as the phase for BP measurement. In this embodiment, time sequence of Korotkoff sounds are reversed, that is, the diastolic time t3 comes before the systolic time t1 in a time sequence and the diastolic pressure is determined firstly when the Korotkoff sounds start, and the systolic pressure is determined secondly when the Korotkoff sounds stop. Therefore, when a slow inflation is used for measurement of BP, the diastolic time t3 and systolic time t1 are entered by the operator by pressing one button in time sequence or a diastolic button and a systolic button separately when the Korotkoff sounds start and stop in sequence during the inflation phase of the measurement cycle.

In a preferred embodiment, more than one personal systolic PIP's are determined and averaged. The average personal systolic PIP is stored for future measurement of SBP in said subject. Similarly, more than one personal diastolic PIP's are determined and averaged. The average personal diastolic PIP is stored for future measurement of DBP in said subject.

The invention also provides an apparatus for implementing the new method, the apparatus having a microprocessor unit (MUP), a display unit, a program memory accessible by the MPU, at least one button for an operator to enter auscultatory BP or a signal indicating the time at which an auscultatory BP is determined, a first software program component stored within the program memory for operating the apparatus and for determining oscillometric BP in a subject according to the oscillometric method, a data memory inside or outside of the MPU for storing data from the MPU and storing personal PIP's for a subject, and a second software program component stored within the program memory for determining personal PIP's for a subject.

In one embodiment of the apparatus, one button is provided to enter the systolic time t1 and diastolic time t3 by sending a systolic signal and a diastolic signal in time sequence.

In another embodiment, two buttons are provided, one for entering the systolic time t1 by sending a systolic signal and the other for the diastolic time t3 by a diastolic signal.

In another embodiment, two buttons are provided, one for increasing and the other for decreasing a pressure value displayed on the display unit, and both are, when operated at the same time, for entering into the MPU a displayed pressure value as an auscultatory systolic or diastolic BP.

In a further embodiment, three buttons are provided, one for increasing, one for decreasing a pressure value displayed on the display unit, and the third one for entering into the MPU a pressure value displayed on the display unit as an auscultatory systolic or diastolic BP.

In a preferred embodiment of the apparatus, the program memory is a ROM or EEPROM and the data memory may be any suitable storage means such as a RAM, EEPROM, or a disc drive. The first and second software program components may conveniently be contained within a single software program.

### BRIEF DESCRIPTION OF THE DRAWINGS

In drawings which illustrate specific embodiments of the invention, but which should not be construed as restricting the spirit or scope of the invention in any way:

FIG. 1 is a block diagram of an electronic blood pressure monitor with three buttons for communicating with a MPU;

FIG. 2 illustrates the calibration of an electronic blood pressure monitor with three buttons for communicating with the MPU; and

FIG. 3 illustrates a cuff pressure waveform with superimposed pulse signals and the corresponding separated pulses signals and their oscillometric envelope.

### DETAILED DESCRIPTION OF EMBODIMENT

The new invention provides apparatus for BP measurement and a method for calibrating the apparatus for a given subject. Said apparatus includes hardware and software. Said hardware includes an inflatable part such as a cuff, an inflation part such as a pump, a valve for deflating the inflatable part, a pressure sensor for measuring the pressure in said inflatable part, electronic circuits for conditioning the pressure sensor signal, a display such as a LCD for display pressure and other relevant information, a microprocessor unit (MPU) for all controlling, calculation and communication functions of the apparatus, and at least one button for communicating with the MPU during the process of calibrating the apparatus for a given subject. Said software includes an oscillometric BP measurement software component and a personal PIP determination software component. Said electronic BP monitor may further include a calibration selection switch to select between standard PIP's and personal PIP's for use in BP measurement.

Said button may be an electro-mechanical switch with a plastic cap on the top to form a button switch. Such a button switch may be either in a normally “off” position or a normally “on” position. When pressed, the button switch changes from either the off position to the on position or from the on position to the off position, sending to the MPU a signal which is change of electrical levels either from “low” to “high” or from “high” to “low”. The time of electrical level change is the time the button is pressed. Therefore, software in the MPU may determine the time when the button is pressed according to the time the signal changes electrical levels. The signal may also be used to change the displayed value. For instance, when the button is pressed five times, the MPU may increase or decrease the displayed pressure value by five mmHg.

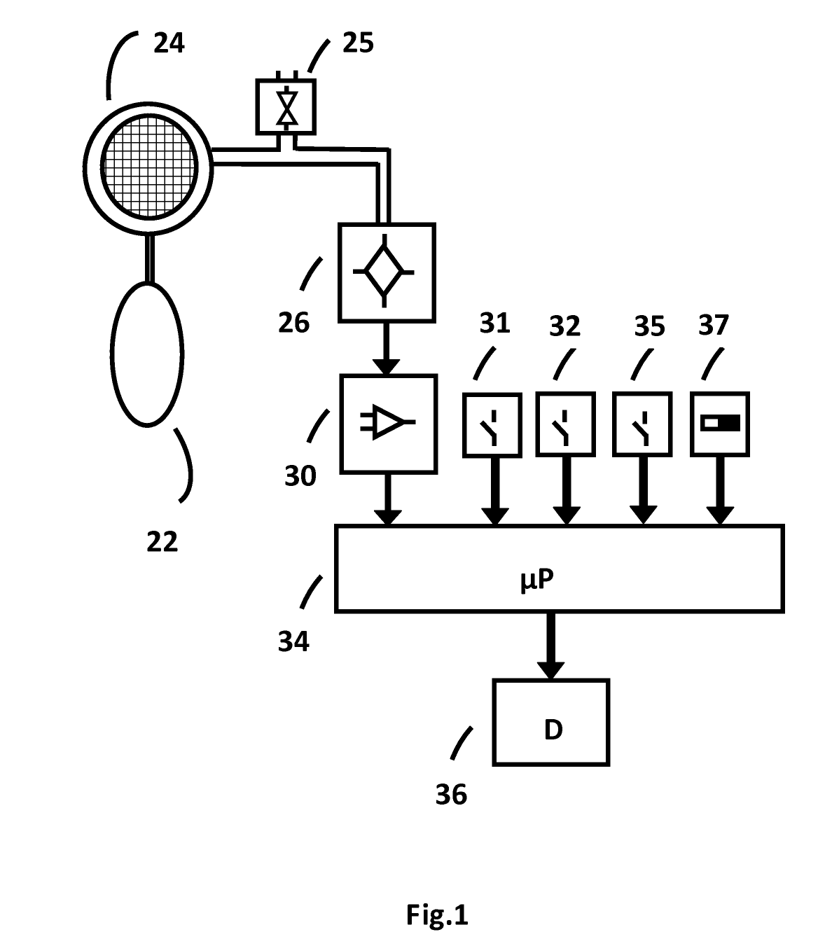

FIG. 1 shows one embodiment of the present invention, in which an electronic blood pressure monitor includes an inflation pump 22; inflatable cuff 24; deflation valve 25; pressure sensor 26, differential amplifier 30, pressure increase button 31, pressure decrease button 32, power switch 33. MPU 34, pressure input button 35, LCD display 36, and calibration selection switch 37. The inflation cuff 22 may be a hand pump or an electrical pump. The inflatable cuff 24 may be an upper arm cuff. The deflation valve 25 may be a hand-controlled valve or an electromagnetic valve controlled by the MPU. The pressure sensor 26 and differential amplifier 30 may be separate parts or an integrated part, and may be replaced with a capacitance type of sensor and an oscillator. The LCD display 36 may be replaced by other display types such as LED's or a graphical display. The calibration selection switch 37 may be a two-position switch, one position being used to indicate that the standard PIP's will be used in BP measurement and the other position to indicate that the personal PIP's previously obtained for a subject will be used. When the calibration selection switch 37 is at the position for the standard PIP's, the calibration selection switch 37 may send a low level electrical signal to the MPU 34. When the calibration selection switch 37 is at the position for the personal PIP's, the calibration selection switch 37 may send a high level electrical signal to the MPU 34. The calibration selection switch 37 may also be a multiple position switch for positioning to more than one set of personal PIP's so that the BP monitor may be calibrated for more than one subject.

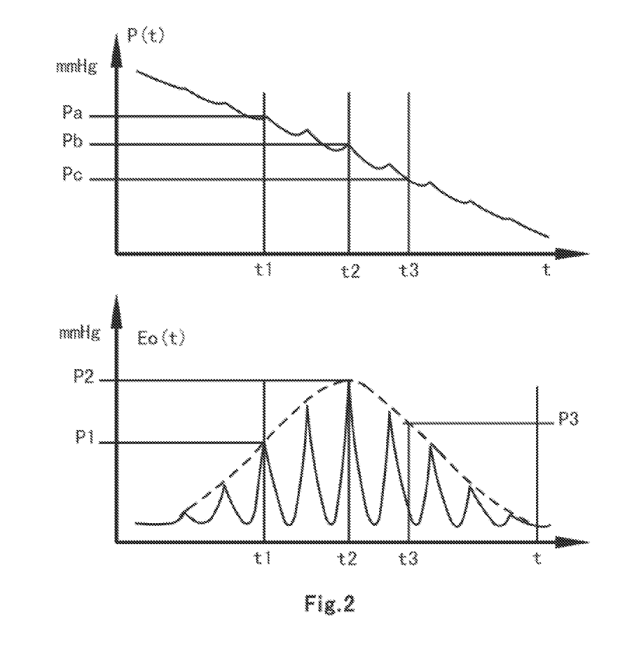

As shown in FIG. 2, calibration of the electronic BP monitor for a given subject involves the use of the BP monitor 38 as well as a stethoscope 20 by an operator (not shown) to measure the BP of the subject simultaneously with both the oscillometric method implemented in the BP monitor 38 and with the auscultatory methods by the operator. In one embodiment. the calibration is done in the following steps: 1) start the BP monitor by pressing the power switch 33; 2) place the sound sensing part of the stethoscope 20 on the artery below the cuff 24 to detect Korotkoff sounds; 3) while the BP monitor 38 is measuring the BP of the subject automatically with the oscillometric method; the operator simultaneously measures the BP of the subject with the auscultatory method by listening to the Korotkoff sounds and watching the pressure display of the BP monitor 38 or a separate pressure meter such as a mercury sphygmomanometer (not shown) connected to the BP monitor 38's cuff 24 with a three way air connector (not shown); 4) after the BP measurement is completed with both the measurement methods; the oscillometric systolic and diastolic pressure are displayed on the display 36; 5) select the displayed systolic or diastolic pressure for input by pressing the pressure input button 35 any number of times to switch between the displayed systolic or diastolic pressure, the selected pressure being displayed in a flashing manner which may be at a rate of flashing once per second; 6) press the pressure increase button 31 to increase or the pressure decrease button 32 to decrease the selected pressure value which is displayed in a flashing manner until the selected pressure value is the same as the pressure value obtained by the auscultatory method; 7) enter the selected value into the MPU by pressing the pressure input button 35, which also change the selected pressure from systolic to diastolic or from diastolic to systolic pressure; 8) repeat the steps 5), 6) and 7) until both the auscultatory systolic and diastolic pressures are entered into the MPU; 9) wait for two minute without action on the three buttons 31, 32 and 35; 10) after the two minutes time has passed, the MPU will take the input values of the auscultatory systolic and diastolic pressure and determine the personal systolic and diastolic PIP's for the subject and store them in a memory area for personal PIP's.

Next time when the BP monitor 38 is turned on, if the calibration selection switch 37 is at the personal PIP's position, the personal PIP's instead of the standard PIP's will be used in blood pressure measurement.

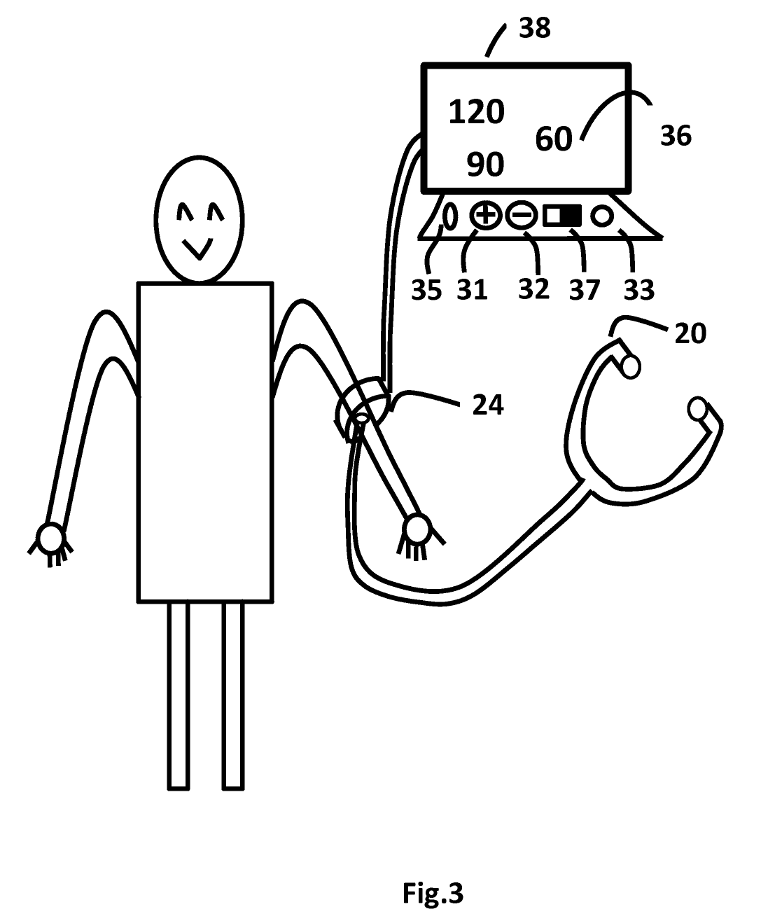

There is a software program component for carrying out the oscillometric BP measurement and a software program component for carrying out the auscultatory systolic and diastolic pressure input and for carrying out the determination of the personal PIP's embedded in the MPU 34 shown in FIG. 1. The two software program components may he separate parts or one integrated part. As shown in FIG. 3, a cuff pressure waveform P(t) and an oscillometric envelope waveform E0(t) shown as the dashed line are obtained by the oscillometric BP measurement software program component. When an auscultatory systolic pressure is entered by the operator, under the control of the personal PIP determination software program component, the MPU determines the personal systolic PIP as follows: 1) find the time t1 at which the auscultatory systolic pressure equals to the cuff pressure Pa on the waveform P(t); 2) find the oscillometric pressure P1 at time t1; and 3) calculate the ratio between P1 and the peak value P2 of the oscillometric envelope; 4) store the ratio P1/P2 as the personal systolic PIP. Similarly, the MPU determines the personal diastolic PIP as follows: 1) find the time t3 at which the auscultatory diastolic pressure equals to the cuff pressure Pc on the waveform P(t); 2) find the oscillometric pressure P3 at time t3; and 3) calculate the ratio between P3 and the peak value P2 of the oscillometric envelope; 4) store the ratio P3/P2 as the personal diastolic PIP.

In another embodiment, the pressure input button 35 shown in FIG. 2 may be eliminated and its function may be replaced by the combination of the pressure increase button 31 and the pressure decrease button 32. When the pressure increase button 31 and the pressure decrease button 32 are pressed simultaneously, they perform the function of pressure input button 35.

In a preferred embodiment, the pressure increase button may function as the systolic time input button and the pressure decrease button may function as the diastolic time input button. During the cuff deflation phase in BP measurement with the BP monitor 38 shown in FIG. 2, when the operator hears the start of Korotkoff sounds, the operator may press the pressure increase button 31 immediately, sending a signal to the MPU, which then records the time of the signal as the auscultatory systolic pressure time t1 as shown in FIG. 3. Similarly, when the operator hears the stop of Korotkoff sounds, the operator may press the pressure increase button 31 or the pressure decrease button 32 immediately, sending a signal to the MPU, which then records the time of the signal as the auscultatory diastolic pressure time t3 as shown in FIG. 3. After the auscultatory systolic and diastolic time t1 and t3 have been determined, the personal PIP's may be determined in the same way as described above. in this embodiment, either one calibration button only or two calibration buttons may be used.

In another preferred embodiment. the calibration process is repeated multiple times to obtain multiple systolic personal PIP's and diastolic personal PIP's. Then the multiple systolic personal PIP's are averaged to obtain an average systolic personal PIP and store in memory for personal use in future measurement of systolic BP of the subject. Similar, the multiple diastolic personal MP's are averaged to obtain an average diastolic personal PIP and store in memory for personal use in future measurement of diastolic BP of the subject. Preferably, three systolic personal PIP's are averaged and three diastolic personal PIP's are averaged.

Accordingly, while this invention has been described with reference to illustrative embodiments, this description is not intended to be construed in a limiting sense. Various modifications of the illustrative embodiments, as well as other embodiments of the invention, will be apparent to persons skilled in the art upon reference to this description, it is therefore contemplated that the appended claims will cover any such modifications or embodiments as fall within the true scope of the invention.

1. A method for calibrating an electronic blood pressure monitor for personal use for a given subject, said method comprising the steps of:
a) Determining a personal PIP for said given subject; and
b) Storing said personal PIP in said blood pressure monitor for use in future measurement of blood pressure in said subject.

2. A method as claimed in 1 wherein said step of determining a personal PIP comprises the steps of
a) Completing a blood pressure measurement cycle on said subject with said blood pressure monitor;
b) Inputting a blood pressure value obtained simultaneously with the auscultatory method by an operator;
c) Determining the time at which the cuff pressure is the same as said blood pressure value obtained simultaneously with the auscultatory method by said operator;
d) Determining the oscillometric envelope value P at said time;
e) Calculating the ratio between said oscillometric envelope value P and the oscillometric envelope peak value P2; and
f) Setting said ratio as a personal PIP of said subject.

3. A method as claimed in 1 wherein said step of determining a personal PIP comprises the steps of
a) Completing a blood pressure measurement cycle on said subject with said blood pressure monitor;
b) While in the deflation phase of the blood pressure measurement cycle, determining the time at which a blood pressure is obtained with the auscultatory method by an operator;
c) Determining the oscillometric envelope value P at said time;
d) Calculate the ratio between said oscillometric envelope value P and the oscillometric envelope peak value P2 and
e) Setting said ratio as a personal PIP of said subject.

4. A method as claimed in 1 wherein said personal PIP is one of a personal systolic PIP and a personal diastolic PIP.

5. A method as claimed in 4 wherein said method further comprises the steps of
a) Repeating the same steps as in claim 4) to obtain multiple personal PIPs;
b) Averaging said multiple personal PIPs to obtain an average personal PIP; and
c) Storing said average personal PIP in said blood pressure monitor for use in future measurement of blood pressure of said subject.

6. A method as claimed in 1 wherein said step of determining a personal PIP comprises the steps of
a) Completing a blood pressure measurement cycle on said subject with said blood pressure monitor using a slow inflation phase as the blood pressure measurement phase;
b) While in the inflation phase of the blood pressure measurement cycle, determining the time at which a blood pressure is obtained with the auscultatory method by an operator;
e) Determining the oscillometric envelope value P at said time;
d) Calculate the ratio between said oscillometric envelope value P and the oscillometric envelope peak value P2 and
e) Setting said ratio as a personal PIP of said subject.

7. An electronic blood pressure monitor capable of being calibrated for a given subject, said blood pressure monitor comprising:
a) An inflatable cuff for occluding an artery of said given subject;
b) A pressure sensor for measuring the pressure in said inflatable cuff;
c) A display for displaying pressure values measured with said pressure sensor;
d) At least one button for an operator to input to said blood pressure monitor one of a signal indicating the time at which a blood pressure is obtained by said operator with the auscultatory method and a blood pressure value obtained by said operator with the auscultatory method; and
e) A MPU for communicating with said at least one button for determining a personal PIP of said subject according to said one of a signal and a blood pressure value, said personal PIP of said subject being used specifically for measurement of blood pressure of said subject.

8. An electronic blood pressure monitor as claimed in 7 wherein said personal PIP is one of a personal systolic PIP and a personal diastolic PIP.

9. An electronic blood pressure monitor as claimed in 7, comprising one button for sending to said MPU in sequence a systolic signal indicating the time at which a systolic blood pressure is obtained and a diastolic signal indicating the time at which a diastolic blood pressure is obtained by an operator with the auscultatory method.

10. An electronic blood pressure monitor as claimed in 7, comprising two buttons, one for sending to said MPU a systolic signal indicating the time at which a systolic blood pressure is obtained and the other one for sending to said MPU a diastolic signal indicating the time at which a diastolic blood pressure is obtained by an operator with the auscultatory method.

11. An electronic blood pressure monitor as claimed in 7, comprising two buttons, one, when operated alone, for increasing a pressure value displayed in said display, the other, when operated alone, for decreasing a pressure value displayed in said display, and both, when operated together, for entering into said MPU in sequence a systolic pressure and a diastolic pressure displayed in said display.

12. An electronic blood pressure monitor as claimed in 7, comprising three buttons, one for increasing a pressure value displayed in said display, another one for decreasing a pressure value displayed in said display, and a third one for entering into said MPU the pressure displayed in said display.
